(19) United States
(12) Patent Application Publication
(10) Pub. No.: US 2025/0232833 A1
(43) Pub. Date: Jul. 17, 2025

(54) Cyclin D1 Based Cancer Vaccine

(71) Applicant: Anders Kaare Nørgaard, Glostrup (DK)

(72) Inventors: Anders Kaare Nørgaard, Glostrup (DK); Morten Nielsen, Holte (DK)

(21) Appl. No.: 18/412,484

(22) Filed: Jan. 13, 2024

(51) Int. Cl. G16B 15/30; G16H 20/17

(52) U.S. Cl. CPC G16B 15/30; G16H 20/17

(57) ABSTRACT

The present disclosure provides a vaccine against cancers that express both high levels of Cyclin D1 and MHC-II. That includes hormone positive breast cancers and some prostate cancers and melanoma. Cyclin D1 is not presented well on MHC-I, and it expresses very little on non-cancerous tissues, so that it is possible, based on a genomic test of the individual, to design a polytope based on epitopes from Cyclin D1 that in most cases display on MHC-II and not on MHC-I, such that the cancer after vaccination with the polytope will reveal itself to the immune system and get eradicated while there are little side effects in that the vaccine will not make the immune system harm the healthy tissue. Since the invention utilizes the fact that the cancer expresses MHC-II, it may work with other antigens than Cyclin D1 as long as the cancer expresses MHC-II.

### BACKGROUND/SUMMARY

### Background of the Invention

The present invention relates to the use of the immune system of a human to recognize and kill a disease like cancer—in the following “the cancer”. In the following, key functions related to the functioning of the immune system are presented, in order to explain the background of invention, and not in order to explain the immune system to a person not acquainted with it.

Cancers are the cause of death of many humans, and most cancers are not completely eradicated once they develop, in that current treatments rarely use the immune system to eradicate them. They may in some cases use the immune system to make the cancer treatments more effective e.g. by making T cells more effective through checkpoint inhibition treatments, which dominate current immunology treatments for cancer. But the previous uses of the immune system rarely makes it remove the cancer completely once it is discovered, or prevent it from developing, as it can do with other diseases. One of the causes is that a cancer develops in cells that express the same on the surface to the immune system, as healthy cells do, and the immune system through its functionality—e.g. through its selection of T cells—is barred from attacking healthy cells (in order to prevent autoimmune diseases from happening).

In order to elicit an immune response the cancer must be recognized on either the MHC-I or the MHC-II. It is done by the presentation of epitopes, which are a part of an antigen, wrapped into an MHC molecule. This complex of an epitope wrapped into MHC serves as a Ligand for the T Cell Receptor (TCR), such that if the said Ligand matches a TCR of a T cell, then said T cell is attracted and its activation is initiated.

The functioning and appearance of the MHC-I and MHC-II molecules are determined by the following genes:
MHC-I: HLA-A, HLA-B, HLA-C; and of without significance for interaction with T cells: HLA-E, HLA-F, HLA-G, and HLA-H.
MHC-II: HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, and HLA-DRB5.

The selection of T cells in the thymus ensures that the matching to a TCR will not be likely, if the protein is naturally occurring in the tissue above a certain threshold (except in the event of an autoimmune disorder).

T cells of the type CD8+—Cytotoxic T Lymphocytes (CTL)—are potentially bound to MHC-I, i.e. to Ligands consisting of an epitope wrapped into an MHC-I molecule. The CD8+ CTLs may then kill the cell that presents the said Ligand.

T cells of the type CD4+—Helper T cells (Th), and potentially other CD4+ T cells and CD4+ cells that are themselves cytotoxic (CD4+ CTLs)—are potentially bound to MHC-II, i.e. to Ligands consisting of en epitope wrapped into an MHC-II molecule. The fact that CD4+ cells can be cytotoxic themselves is a rather new discovery, reported in the papers [13], [24], [25], [26], [27], and [28], but not reflected in most overviews of the CD4+ phenotypes, and currently a CTL is often assumed to be a CD8+ T cell.

The CD4+ cells may therefore do one of several things: (a) They may attract CD8+ cytotoxic T cells, which in turn kill the cell that presents the said Ligand consisting of an epitope wrapped into MHC-II, (b) they may directly kill the cell (being a CD4+ cytotoxic T cell), (c) they may through the secretion of cytokines attract B cells that directly or through antibodies that they produce kill the cell with the Antigen, or (d) some other immune reaction. It is likely that some significant immune reaction happens, i.e. one or several of (a), (b), (c), and (d), since it seems that some MHC-II gene alleles have a strong influence on the occurrence of and are in some cases even protective for breast cancer according to the papers [14], [15], [16], [17], [18], [19], and [20]. Whether the immune reaction consists of one or a combination of the above mechanisms-including whether there exist further mechanisms elicited by MHC-II and CD4+ T cells-does not affect the present invention.

There is a growing interest in using MHC-II, either as a supplement to MHC-I (“activating both arms of the immune system”) or alone.

All cells with a nucleus (that is all cells of the human body with a few exceptions like red blood cells) have the ability to express MHC-I on their surface.

Only cells of the type “professional Antigen Presenting Cells”, sometimes just called “Antigen Presenting Cells”, (APC) and some cancer cells have the ability to express MHC-II on their surface. The APCs include Dendritic Cells (DCs), Macrophages, and B-cells. The fact that some cancers can present MHC-II is a rather new discovery, not reflected in most overviews, and it is generally assumed that MHC-II is only present on the surface of APCs. The cancers that can express MHC-II include
breast cancer (BC),
prostate cancer (PCa),
melanoma,
colorectal cancer,
ovarian cancer,
classic Hodgkin lymphoma,
glioma, and
non-small cell lung cancer (NSCLC).
   We are currently not sure that all of MHC-II is expressed on these cancers, and we are not sure that all variants of the above cancers express MHC-II.

Proteins are cut into epitopes before being attempted to be wrapped into an MHC molecule. The said epitope usually has the length 9, spanning from 8 to 14 (amino acids), in MHC I and the length 15, spanning from 13 to 25 (amino acids), in MHC-II. Then these epitopes can be presented, wrapped into MHC, on the surface of the cell, if they pass a number of further steps in the Antigen Presentation Pathway (APP), of which the wrapping with the MHC molecule is one step.

The proteins that have the possibility to be cut into epitopes that after being wrapped into an MHC molecule may be presented to and match a TCR of a T cell, and thus trigger an immune response, are called Antigens. Antigens are thus either (1) purely external (e.g. pathogens that represent a disease), (2) mutated proteins that haven't been present prior to the mutation, or (3) proteins that are expressed in an amount that is significantly above normal.

Tumor Specific Antigens (TSAs) are—mostly—mutated proteins (i.e. generated by a gene that is mutated). They are thus very immunogenic candidates of Antigens in that they are not associated with a normal “healthy” level (of expression), and TSAs are thus the focus of much of the cancer vaccine effort. But TSAs come with two main problems: (1) To capture them, a sample of the tumor is needed and must be extracted e.g. as a biopsy, and (2) since they come into existence due to a mutation, it is likely that the cancer can mutate again, away from the protein, and thus the cancer can live on in cells that do not express the TSA. In any case the vaccine requires the cancer to be there, so it can only be therapeutic, not preventative.

Tumor Associated Antigens (TAAs) are examples of proteins that have a lower level of expression in normal tissue and a higher level in cancer tissue. The fact that cells with normal levels of these Antigens still express them, means that there is either a threshold mechanism, or an accepted low level of killing of healthy cells, or they will be ineffective at any level as Antigens, because the selection of T cells in the thymus will make it hard to find a T cell whose TCR matches the Antigen. This mechanism is not causally determined, only empirically. In any event the effectiveness of a TAA as a vaccine candidate depends on whether there is a wide distance between how much it is expressed in the cancer on the one side and how much it is expressed in normal tissue—so that an appropriate amount of vaccine can be determined in order to “teach” the immune system to kill the cancer cells and not the healthy tissue. “Teach” in this case means adjusting (enhancing) the number of T cells so that they will leave the healthy tissue alone or not substantially kill it.

All cells (with a nucleus) use the presentation of epitopes from their proteins on MHC-I. This includes the APCs and all the cancer cells as well as all other cells (with a nucleus). See FIG. 6.

The APCs can swallow and digest exterior Antigens (a process called phagocytosis), so that they are presented on the MHC-II (and the MHC-I) of the said APCs, together with the internal proteins of the said APCs (cut into epitopes and wrapped into MHC-II).

Some cancer cells can present their internal proteins and thus Antigens, cut into epitopes, on MHC-II (the cancer cell types mentioned above).

The focus of prior art has traditionally been on MHC-I mechanisms for cancer vaccines and immunotherapy for cancer in general. This can be because there is a simpler mechanism behind this kind of immunity against cancer, with CD8+ CTLs doing the killing directly, not being aware that this is the case for MHC-II as well. Much of the focus has then been on (1) turning “cold” tumors into “hot” tumors (helping the MHC-I function of making the cancer visible to CD8+ CTLs) and on checkpoint inhibition of these CD8+ CTLs (avoidance of two mechanisms that can slow down or inactivate CD8+ CTLs: The PD-1 and CTLA-4 receptors).

The recent focus of prior art has furthermore been on TSA, not TAA. This focus on TSA has the following drawbacks:
The cancer may mutate away from the specific antigen.
The antigen may not represent the full cancer.
There may be errors in the extraction and detection process

Cyclin D1 (having the gene code and abbreviation CCND1) is an example of a TAA, in that it is expressed in elevated amounts in among others the following cancers:
Breast cancer (in particular the hormone positive BCs, the socalled ER+ BCs, which constitute more than half the BCs, cf. FIG. 11 and FIG. 17),
prostate cancer (cf. FIG. 11 and FIG. 18),
melanoma (cf. FIG. 11 and FIG. 19),
liver cancer,
renal cancer, and
urothelial cancer

Cyclin D1 and MHC-II are thus both expressed on the following cancers:
Breast cancer (ER+ BCs),
prostate cancer, and
melanoma.

Other TAAs than Cyclin D1 (CCND1) include: Mucin-1 (MUC1), Carcinoembryonic antigen (CEA), Human Epidermal Receptor 2 (HER2 or ERBB2), Telomerase Reverse Transcriptase (TERT), Wilms Tumor Gene (WT-1), Sialyl-Tn, Myc proto-oncogene protein (MYC), Prostate-specific membrane antigen (PSMA), Prostatic Acid Phosphatase (PAP), Prostate Specific Antigen (PSA), and Cancer/testis antigen 1 (NY-ESO-1).

Cyclin D1 is presented more weakly on MHC-I than on MHC-II compared to other TAAs cf. FIG. 10.

Probably because of this fact as well as the prevalent focus on MHC-I the Antigen Cyclin D1 is rarely listed in overviews of potential TAAs. Overviews present other TAAs like MUC1, CEA, HER2, TERT etc.

Cyclin D1 is well differentiated i.e. highly expressed in cancerous tissue compared to normal tissue compared to other TAAs cf. FIG. 11 and FIG. 14.

Cancers often have the ability to silence the MHC-I, but leaving MHC-II untouched for those cancers, which express MHC-II. Taking this fact together with the fact that Cyclin D1 already is more poorly presented on MHC-I than on MHC-II means that whenever an expression of a Cyclin D1 epitope surfaces—in particular when it surfaces on MHC-II, it is most likely from a cancer cell (with the exception of a weak expression on the APCs).

It may explain why some MHC-II alleles are protective of breast cancer as reported in [14], [15], [16], [17], and [18], the same alleles producing an MHC-II molecule seemingly good at binding Cyclin D1. So this fact suggests that if we elevate the ability of CD4+ T cells in the rest of the cases to “see” the Cyclin D1 epitopes, then in those cases the breast cancer, and maybe prostate cancer and melanoma, may trigger an immune response that kills them.

The area of cancer vaccine comprises as a key step to predict whether a protein under consideration as parts of an Antigen or a subset of it, called an epitope, will pass through an important step of the APP being whether the Antigen is able to be cut into epitopes and one or several of these epitopes will bind to MHC and then possibly be presented on the surface of the cell. This analysis depends on the allele of the MHC genes—in MHC-I the genes named HLA-A, HLA-B, HLA-C, and to some extent the other MHC-I genes—and in MHC-II the genes named HLA-DPA1, HLA-DPB1, HLA-DQA1, HLA-DQB1, HLA-DRA, HLA-DRB1, HLA-DRB3, HLA-DRB4, HLA-DRB5, and to some extent the other MHC-II genes. This prediction is carried out as a computer based program of which one is available as the services [S4] for MHC-I and [S5] for MHC-II. It is known that if this prediction shows that an epitope can not bind to the MHC in question, then it will not show on the surface of the cell. So the binding of epitopes is a necessary condition for them to be shown.

This prediction analysis is carried out either based on knowledge of the alleles of the human being considered to have the vaccine or on a significant set of alleles that appear with a significant frequency in the human population.

This prediction analysis calculation is deemed to have converged to precision or optimality, but it is likely to become updated in the future to become better. For an allele of either MHC-I [S4] or MHC-II [S5] each possible epitope of a protein is associated with a score that puts the said epitope in the category of being either
A “non-binder”,
a “weak binder” (WB), or
a “strong binder” (SB)

“Strong binders” are deemed statistically to almost certainly bind to the corresponding MHC molecule and thus possibly move to the cell surface and display to the immune system, “weak binders” can maybe bind to the MHC molecule, and “non-binders” are certain not to bind to the MHC molecule and will therefore not display at the cell surface to the immune system.

Multiple selected epitopes can be combined into a so-called polytope by adding them as chains on a string potentially introducing linker amino acids.

The area of cancer vaccine further comprises a vaccine technology where the selected polytope is used as a vaccine agent e.g. by using its digital equivalent transformed into mRNA as input to the generation of a part of the vaccine agent. The FDA approved mRNA vaccines for COVID-19, and this method is frequently being used in clinical trials today, cf paper [21]. Other vaccine methods beyond mRNA comprise
SAM: Self Amplifying mRNA vaccine
LNP mRNA: Lipid nanoparticles mRNA vaccine
DC (Dendritic Cell) vaccine
ISV: In situ vaccine

The epitopes of an Antigen that bind to an MHC molecule and thus potentially are presented to the immune system vary a lot for different HLA alleles. This means that the Position in the protein that represents the antigen from which a “strong binder” epitope exists varies a lot depending on the allele. For the Antigen Cyclin D1, having a protein length of 295 (amino acids), the number of MHC-II “strong binders” according to the prediction service [S5] varies depending of the selected MHC-II from none (0) to 11, with most in the bracket of 1-8. And less “strong binders” in MHC-I. Any human expresses up to six different MHC-I and up to 12 different MHC-II molecules, and despite the fact that a molecule has no “strong binder”, then another will most likely have a strong binder. We have computed an estimate of the share of the world's population based on the service in [S7], which seems according to [S5], that have no MHC-II strong binders among the epitopes of Cyclin D1 given their allele combination, to be less than 1% (0.3%).

It is therefore in most cases (for Cyclin D1) possible to identify one or several epitopes that bind strongly to MHC-II and not at all to MHC-I. It may be that a “strong binder” to MHC-I is fully embedded in a “strong binder” to MHC-II, and it is therefore discarded (omitted in the final polytope).

The following services and databases are used:

| [#] | Service | URL | Description |
|---|---|---|---|
| S1 | Tantigen | http://projects.methilab.org/ | A database of antigens. |
| | | tadb/HTML/search.php | The Cyclin D1 record in the database: |
| | | | http://projects.methilab.org/tadb/cgi/ |
| | | | displayAntigen.pl?ACC=Ag000285 |
| S2 | Human | https://v17.proteinatlas.org/ | A database of proteins and cancers. It is linked |
| | Protein | | to from Tantigen. |
| | Atlas | | The Cyclin D1 record in the database: |
| | | | https://v17.proteinatlas.org/ |
| | | | ENSG00000110092-CCND1/pathology |
| S3 | Antibodypedia | https://www.antibodypedia.com/ | A database of antibodies with further |
| | | | descriptions of the antigen and the list of |
| | | | antibodies to it. |
| | | | The Cyclin D1 record in the database: |
| | | | https://www.antibodypedia.com/gene/3660/CCND1 |
| S4 | MHC I | https://services.healthtech.dtu.dk/ | An online service provided by the Danish |
| | binding | services/NetMHCpan-4.1/ | Technical University (DTU) fronting an |
| | prediction | | application that can combine (1) most alleles of |
| | | | the genes behind MHC-I with (2) any protein in |
| | | | order to predict the ability to bind epitopes of |
| | | | said protein to MHC-I |
| S5 | MHC II | https://services.healthtech.dtu.dk/ | An online service provided by DTU fronting an |
| | binding | services/NetMHCIIpan-4.3/ | application that can combine (1) most alleles |
| | prediction | | (some of them coupled into haplotypes) of the |
| | | | genes behind MHC-II with (2) any protein in |
| | | | order to predict the ability to bind epitopes of |
| | | | said protein to MHC-II |
| S6 | Pop Cover | https://services.healthtech.dtu.dk/ | An online service provided by DTU fronting an |
| | | services/PopCover-2.0/ | application that can construct an optiumum |
| | | | peptide for the world population taking into |
| | | | account allele frequencies, i.e. without making |
| | | | a personal genomic test |
| S7 | Allele | https://allelefrequencies.net/hla.asp | Allele frequencies (of the HLA genes) id |
| | Frequencies | | different parts of the World's population |

The allele frequencies of the World population can be deduced from [S7]. It includes, at the top (for information):

### MHC-I:

HLA-A-ordered with descending likelihood, top 5 shown:
HLA-A02: 01 25%
HLA-A01: 01 12%
HLA-A03: 01 12%
HLA-A24: 02 10%

HLA-B-ordered with descending likelihood, top 5 shown:
HLA-B07: 02 10%
HLA-B08: 01 8%
HLA-B15: 01 7%
HLA-B44: 02 5%
HLA-B35: 01 5%

HLA-C-ordered with descending likelihood, top 5 shown:
HLA-C07: 01 3%
HLA-C07: 02 3%
HLA-C04: 01 3%
HLA-C06: 02 3%
HLA-C12: 03 2%

Leading to the likelihood of having the MHC-I combinations (assuming that there is no cross-coupling or covariance, normally attributed to haplotypes)—ordered with descending likelihood, top 5 shown:
HLA-A02: 01 HLA-B07: 02 HLA-C07: 01 0.079%
HLA-A02: 01 HLA-B07: 02 HLA-C07: 02 0.078%
HLA-A02: 01 HLA-B07: 02 HLA-C04: 01 0.070%
HLA-A02: 01 HLA-B08: 01 HLA-C07: 01 0.062%
HLA-A02: 01 HLA-B08: 01 HLA-C07: 02 0.062%

There are at least 9,000 such allele combinations on MHC-I based on a very limited sample of allowable alleles (HLA-B representing 89% of the world's population, HLA-C representing only 23% of the world's population).

### MHC-II:

HLA-DPx-haplotypes ordered with descending likelihood, top 5 shown:
HLA-DPA102: 02-DPB105: 01 21%
HLA-DPA101: 03-DPB104: 01 15%
HLA-DPA101: 03-DPB102: 01 14%
HLA-DPA101: 03-DPB104: 02 10%
HLA-DPA101: 03-DPB103: 01 3%

HLA-DQx-haplotypes ordered with descending likelihood, top 5 shown:
HLA-DQA105: 01-DQB103: 01 9%
HLA-DQA103: 01-DQB103: 02 9%
HLA-DQA101: 02-DQB106: 02 8%
HLA-DQA101: 01-DQB105: 01 8%
HLA-DQA105: 01-DQB102: 01 7%

HLA-DRx-ordered with descending likelihood, top 5 shown:
DRB1_07: 01 10%
DRB1 15:01 8%
DRB1_03: 01 7%
DRB1_11: 01 6%
DRB1 01:01 5%

Leading to the likelihood of having the MHC-II combinations (assuming that there is no further cross-coupling)-ordered with descending likelihood, top 5 shown:
HLA-DPA10202-DPB10501 HLA-DQA10501-DQB10301 DRB1_0701 0.18%
HLA-DPA10202-DPB10501 HLA-DQA10301-DQB10302 DRB1_0701 0.17%
HLA-DPA10202-DPB10501 HLA-DQA10102-DQB10602 DRB1_0701 0.17%
HLA-DPA10202-DPB10501 HLA-DQA10101-DQB10501 DRB1_0701 0.16%
HLA-DPA10202-DPB10501 HLA-DQA10501-DQB10301 DRB1 1501 0.14%

There are at least 1,195,000 such allele combinations on MHC-II.

The likelihoods of MHC-I and MHC-II can then be multiplied together.

U.S. Patent Documents

| Pat no | Date | Title | Assignee | About |
|---|---|---|---|---|
| 10,822,389 | 2019 Oct. 29 | Antibody/T-cell Receptor | Eureka, CA | Exogenous |
| | | Chimeric Constructs | | high-affinity |
| | | And Uses Thereof | | TCRs introduced |
| | | | | into the patients |
| 11,421,013 | 2016 Oct. 21 | Antibody/T-cell Receptor | Eureka, CA | [Same] |
| | | Chimeric Constructs | | |
| | | And Uses Thereof | | |
| 11,505,599 | 2017 Jan. 13 | T Cell Receptor-like | MSKCC, NY + | Foxp3 |
| | | Antibodies Specific For | Eureka, CA | peptide/HLA-A2. |
| | | Foxp3-derived Peptides | | T reg cells. |
| | | | | [Same as below] |
| 10,815,273 | 2018 Mar. 13 | Immunogenic WT-1 | MSKCC, NY | Immunotherapy |
| | | Peptides And Methods | | WT1 cancers |
| | | Of Use Thereof | | [Same as below] |
| 11,384,144 | 2017 Nov. 20 | T Cell Receptor-like | MSKCC, NY + | PRAME |
| | | Antibodies Specific For | Eureka, CA | peptide/HLA |
| | | A PRAME Peptide | | class I |
| | | | | [Same as below] |
| 11,242,405 | 2017 Sep. 20 | Monoclonal Antigen- | MSKCC, NY + | HLA-A2 |
| | | binding Proteins To | Eureka, CA | restricted Ras |
| | | Intracellular Oncogene | | peptide |
| | | Products | | [Same as below] |
| 10,239,952 | 2014 Nov. 7 | Anti-WT1/HLA Bi- | MSKCC, NY + | WT1 |
| | | specific Antibody | Eureka, CA | peptide/HLA-A |
| | | | | epitope |
| | | | | [Same as below] |
| 9,919,037 | 2014 Jan. 15 | Immunogenic WT-1 | MSKCC, NY | Immunotherapy |
| | | Peptides And Methods | | WT1 cancers |
| | | Of Use Thereof | | [Same as below] |
| 9,074,000 | 2012 Apr. 2 | T Cell Receptor-like | MSKCC, NY + | HLA-A0201- |
| | | Antibodies Specific For | Eureka, CA | restricted WT1 |
| | | A WT1 Peptide | | peptide |
| | | Presented By HLA-A2 | | [Same as below] |
| 9,926,380 | 2013 Jul. 3 | Monoclonal Antibodies | University of | HLA-A2- |
| | | For Use In Diagnosis | Texas, TX | restricted |
| | | And Therapy Of | | peptide PR-1 |
| | | Cancers And | | |
| | | Autoimmune Disease | | |
| 11,840,562 | 2021 Jun. 17 | Peptides And | Immatics, | Ovarian cancer, |
| | | Combination Of | Germany | MHC-I and II |
| | | Peptides For Use In | | (but not on |
| | | Immunotherapy Against | | cancer) |
| | | Ovarian Cancer And | | (mentions many |
| | | Other Cancers | | antigens but not |
| | | | | Cyclin D1 as an |
| | | | | antigen) |
| 11,464,839 | 2016 Dec. 2 | Methods And Vaccines | Mayo, MN | Anti-cancer |
| | | For Inducing Immune | | vaccine |
| | | Responses To Multiple | | preparations. |
| | | Different MHC | | Mentions CMV, |
| | | Molecules | | MUC1, HER2, |
| | | | | Mesothelin |
| | | | | (MESO), TRAG- |
| | | | | 3, CALR |
| 10,556,943 | 2016 Dec. 16 | HLA-DR Binding | Mayo, MN | Cancer vaccines |
| | | Peptides And Their | University of | via APC: MHC-II |
| | | Uses | Washington, WA | (HLA-DR) (on B- |
| | | | | cells- DCs - not |
| | | | | expressed on |
| | | | | cancers). |
| | | | | Mentions HER2, |
| | | | | CEA, Cyclin D1, |
| | | | | Insulin Growth |
| | | | | Factor Binding |
| | | | | Protein 2 |
| 10,556,965 | 2017 Jan. 27 | Methods And Materials | Mayo, MN | Cancer vaccine, |
| | | For Treating Cancer | | general on how |
| | | | | APCs work with |
| | | | | MHC-II |
| 10,675,358 | 2018 Sep. 24 | Antibody Adjuvant | Stanford + Bolt, | Activate APCs |
| | | Conjugates | CA | re. neoantigens |
| 10,188,712 | 2014 Jul. 30 | Tumor Antigens For | BioNTech + | TNBC, |
| | | Determining Cancer | TRON, Mainz, | vaccination |
| | | Therapy | Germany | generic |
| 11,793,867 | 2018 Dec. 18 | Neoantigens And Uses | BioNTech US, | Neoantigens |
| | | Thereof | MA | |
| 11,650,211 | 2019 Nov. 22 | HLA-based Methods | BioNTech US, | Generic |
| | | And Compositions And | MA | vaccination |
| | | Uses Thereof | | |
| 11,183,272 | 2020 Mar. 19 | Method And Systems | BioNTech US, | MHC-II. |
| | | For Prediction Of HLA | MA | Conflicts with |
| | | Class II-specific | | the DTU service |
| | | Epitopes And | | |
| | | Characterization Of | | |
| | | CD4+ T Cells | | |
| 10,550,166 | 2017 Sep. 12 | Immunogenic Peptides | Commissariat a | Cyclin B1, MHC- |
| | | Of The Cyclin B1 Tumor | l'Energie | II (CD4+) |
| | | Antigen | Atomique et aux | |
| | | | Energies | |
| | | | Alternatives, | |
| | | | France | |

U.S. Patent Applications

| Doc no | Date | Title | Assignee | About |
|---|---|---|---|---|
| US | 2021 Oct. 8 | COMPOSITIONS | [MSKCC, | HLA-A*02- |
| 20230374140 | | TARGETING | NY + | restricted |
| A1 | | NDC80/MHC | Eureka, | NDC80 |
| | | COMPLEXES AND USES | CA] | peptides. CAR- |
| | | THEREOF | | T cells |
| US | 2020 Oct. 4 | MULTI-VALENT | [MSKCC, | Immunotherapy |
| 20220168408 | | IMMUNOTHERAPY | NY + DE] | WT1 cancers |
| A1 | | COMPOSITION AND | | |
| | | METHODS OF USE FOR | | |
| | | TREATING WT1- | | |
| | | POSITIVE CANCERS | | |
| US | 2017 Jan. 13 | T CELL RECEPTOR-LIKE | [MSKCC, | Foxp3 |
| 20190284262 | | ANTIBODIES SPECIFIC | NY + | peptide/HLA- |
| A1 | | FOR FOXP3-DERIVED | Eureka, | A2. T reg cells. |
| | | PEPTIDES | CA] | |
| US | 2018 Mar. 13 | IMMUNOGENIC WT-1 | [MSKCC, | Immunotherapy |
| 20180208626 | | PEPTIDES AND | NY] | WT1 cancers |
| A1 | | METHODS OF USE | | |
| | | THEREOF | | |
| US | 2017 Nov. 20 | T CELL RECEPTOR-LIKE | [MSKCC, | PRAME |
| 20180148503 | | ANTIBODIES SPECIFIC | NY + | peptide/HLA |
| A1 | | FOR A PRAME PEPTIDE | Eureka, | class I |
| | | | CA] | |
| US | 2017 Sep. 20 | MONOCLONAL | [MSKCC, | HLA-A2 |
| 20180134804 | | ANTIGEN-BINDING | NY + | restricted Ras |
| A1 | | PROTEINS TO | Eureka, | peptide |
| | | INTRACELLULAR | CA] | |
| | | ONCOGENE PRODUCTS | | |
| US | 2014 Nov. 7 | Fc-ENHANCED ANTI- | [MSKCC, | WT-1/HLA/A2 |
| 20160369006 | | WT1/HLA ANTIBODY | NY + | antibody |
| A1 | | | Eureka, | |
| | | | CA] | |
| US | 2014 Nov. 7 | ANTI-WT1/HLA BI- | [MSKCC, | WT1 |
| 20160280796 | | SPECIFIC ANTIBODY | NY + | peptide/HLA-A |
| A1 | | | Eureka, | epitope |
| | | | CA] | |
| US | 2014 Jan. 15 | IMMUNOGENIC WT-1 | [MSKCC, | WT1- |
| 20150352201 | | PEPTIDES AND | NY] | expressing |
| A1 | | METHODS OF USE | | cancer |
| | | THEREOF | | |
| US | 2012 Apr. 2 | T CELL RECEPTOR-LIKE | [MSKCC, | HLA-A0201- |
| 20140294841 | | ANTIBODIES SPECIFIC | NY + | restricted WT1 |
| A1 | | FOR A WTI PEPTIDE | Eureka, | peptide |
| | | PRESENTED BY HLA-A2 | CA] | |
| US | 2005 Dec. 22 | Modulation Of Fc Gamma | [NY] | Fc Receptors |
| 20060193857 | | Receptors For Optimizing | | [Innate Immune |
| A1 | | Immunotherapy | | System] |
| US | 2021 Mar. 12 | MULTIPLE INSTANCE | [Germany] | European |
| 20230402126 | | LEARNING FOR | | patent that |
| A1 | | PEPTIDE-MHC | | conflicts with |
| | | PRESENTATION | | DTU service |
| | | PREDICTION | | |
| US | 2021 Jul. 15 | ANTIBODIES AGAINST | Dana | MUC-1 vaccine |
| 20230265208 | | THE MUC1- | Farber, | |
| A1 | | C/EXTRACELLULAR | MA + | |
| | | DOMAIN (MUC1-C/ECD) | Xyone, | |
| | | | MA | |
| US | 2021 Jun. 1 | COMPOSITIONS AND | Dana | HLA class I |
| 20230248814 | | METHODS FOR | Farber, | molecules in |
| A1 | | TREATING MERKEL | MA | MCC |
| | | CELL CARCINOMA | | |
| | | (MCC) USING HLA | | |
| | | CLASS I SPECIFIC | | |
| | | EPITOPES | | |
| US | 2021 Jun. 1 | METHODS FOR | Dana | MHC-I |
| 20230203485 | | MODULATING MHC-I | Farber, | |
| A1 | | EXPRESSION AND | MA | |
| | | IMMUNOTHERAPY | | |
| | | USES THEREOF | | |
| US | 2022 Jun. 10 | DURABLE | [CA] | Neoantigens, |
| 20230047979 | | VACCINATION | | MHC improved |
| A1 | | | | prediction of |
| | | | | binding |
| US | 2022 May 13 | ANTIGEN-BINDING | [CA + | MHC class I, |
| 20230041030 | | PROTEINS TARGETING | MA] | neoantigens |
| A1 | | SHARED NEOANTIGENS | | |
| US | 2020 Sep. 23 | METHODS OF HIGH- | Dana | Using cytokines |
| 20220364079 | | THROUGHPUT | Farber, | to assert which |
| A1 | | IDENTIFICATION OF T | MA | epitopes are |
| | | CELL EPITOPES BY | | expressed well |
| | | CAPTURING | | in both MHC-I |
| | | CYTOKINES ON THE | | and II |
| | | SURFACE OF ANTIGEN- | | |
| | | PRESENTING CELLS | | |
| US | 2020 Oct. 9 | COMPOSITIONS AND | Dana | Reinvigorating |
| 20220363766 | | METHODS FOR | Farber, | MHC-I (for |
| A1 | | TREATING CYTOTOXIC | MA | reinstating |
| | | T CELL RESISTANT | | checkpoint |
| | | TUMORS | | blockade) |
| US | 2020 Jun. 15 | ANTIBODIES AGAINST | Dana | MUC-1 vaccine |
| 20220289863 | | MUC1 AND METHODS | Farber, | |
| A1 | | OF USE THEREOF | MA | |
| US | 2022 Jan. 10 | CHIMERIC RECEPTOR | [MD] | Generic |
| 20220220187 | | THERAPY | | vaccine |
| A1 | | | | |
| US | 2020 Apr. 24 | CANCER THERAPY BY | [UK] | Upragulating |
| 20220118070 | | MODIFYING | | neo-antigens to |
| A1 | | NEOANTIGEN | | be better |
| | | EXPRESSION | | recognized by |
| | | | | MHC-I. |
| US | 2023 May 18 | ARTIFICIAL NUCLEIC | CureVac, | Artificial nucleic |
| 20230399649 | | ACID MOLECULES | Germany | acid molecule |
| A1 | | | | cancer vaccine |
| US | 2019 Dec. 12 | METHODS FOR | Brad Inst, | Neoantigens, |
| 20220062394 | | IDENTIFYING | Dana | MHC-I |
| A1 | | NEOANTIGENS | Farber, MA | |
| US | 2018 Jun. 15 | MATERIALS AND | Mayo, MN | Activating T |
| 20200171170 | | METHODS FOR | | cells, MHC-I |
| A1 | | INCREASING IMMUNE | | |
| | | RESPONSES | | |
| US | 2022 May 26 | COMPOSITIONS AND | Stanford, | MHC-II binding |
| 20220381793 | | METHODS FOR | CA | |
| A1 | | IDENTIFYING MHC-II | | |
| | | BINDING PEPTIDES | | |
| US | 2020 Dec. 23 | EXAMINATION OF | Stanford, | General |
| 20210202029 | | NETWORK EFFECTS OF | CA | immune |
| A1 | | IMMUNE MODULATION | | emulating |
| | | | | software |
| US | 2020 Jul. 30 | Methods And Systems For | Stanford, | MHC-I and II |
| 20210033608 | | Identification Of Human | CA | binding scares, |
| A1 | | Leukocyte Antigen | | calculation |
| | | Peptide Presentation And | | |
| | | Applications Thereof | | |
| US | 2018 Mar. 21 | Antigen Discovery For T | Stanford, | How in general |
| 20200010527 | | Cell Receptors Isolated | CA | T cells |
| A1 | | From Patient Tumors | | recognize MHC |
| | | Recognizing Wild-Type | | (with focus on |
| | | Antigens And Potent | | MHC-I) |
| | | Peptide Mimotopes | | |
| US | 2021 Nov. 11 | MONOCLONAL | BioNTech, | MHC-! and |
| 20230399403 | | ANTIBODIES DIRECTED | Germany | PD-1 |
| A1 | | AGAINST | | |
| | | PROGRAMMED DEATH-1 | | |
| | | PROTEIN AND THEIR | | |
| | | USE IN MEDICINE | | |
| US | 2021 Mar. 30 | TREATMENT | BioNTech, | Generic |
| 20230145774 | | INVOLVING NON- | Germany | vaccination |
| A1 | | IMMUNOGENIC RNA | | |
| | | FOR ANTIGEN | | |
| | | VACCINATION | | |
| US | 2021 Feb. 2 | TREATMENT | BioNTech, | MHC-I and |
| 20230117803 | | INVOLVING ANTIGEN | Germany | PD-1 |
| A1 | | VACCINATION AND | | |
| | | BINDING AGENTS | | |
| | | BINDING TO PD-L1 AND | | |
| | | CD137 | | |

Foreign Patent Documents

| | | | | |
|---|---|---|---|---|
| Doc no | Date | Title | Assignee | About |

| | | | |
|---|---|---|---|
| CN102971003A | 2010 Jun. 2 | Improved cancer therapy | Immatics, |
| | | based on tumor | Tübingen, |
| | | associated antigens | Germany |
| | | derived from cyclin D1 | |
| EP2567707A2 | 2007 Jul. 27 | Composition of tumour- | Immatics, |
| | | associated peptides and | Tübingen, |
| | | related anti-cancer | Germany |
| | | vaccine | |

### OTHER REFERENCES

This list includes papers referenced in this document using [square brackets].

| [#] | URL | Title | Author(s) |
|---|---|---|---|
| 1 | https://journals.aai.org/ | Novel cyclin D1-based DC vaccine | Matthew James Gibb et |
| | jimmunol/article/200/ | inhibits TNBC tumor growth | al., Baylor University, TX |
| | 1_Supplement/181.13/ | | |
| | 60837/Novel-cyclin-D1- | | |
| | based-DC-vaccine- | | |
| | inhibits-TNBC | | |
| 2 | https://www.ncbi.nlm.nih.gov/ | Biological Consequences of Major | Margaret L. Axelrod et |
| | pmc/articles/PMC6467754/ | Histocompatibility Class-II Expression | al., Vanderbilt University, |
| | | by Tumor Cells in Cancer | TN |
| 3 | https://www.ncbi.nlm.nih.gov/ | MHC Class II Binding Prediction-A | Ivan Dimitrov et al., |
| | pmc/articles/PMC2875769/ | Little Help from a Friend | Medical University, Sofia, |
| | | [Not cancer specific; APCs] | Bulgaria |
| 4 | https://www.ncbi.nlm.nih.gov/ | Tumor-Associated Antigens in Breast | Carmen Criscitiello, |
| | pmc/articles/PMC3515784/ | Cancer | European Institute of |
| | | | Oncology, Milan, Italy |
| 5 | https://www.frontiersin.org/ | CIITA-Driven MHC Class II | Roberto S. Accolla et al., |
| | articles/10.3389/ | Expressing Tumor Cells as Antigen | University of Insubria, |
| | fimmu.2019.01806/full | Presenting Cell Performers: Toward | Varese, Italy |
| | | the Construction of an Optimal Anti- | |
| | | tumor Vaccine | |
| 6 | https://www.ncbi.nlm.nih.gov/ | Bioinformatics analysis of the clinical | Guihua Wu et al., |
| | pmc/articles/PMC9543021/ | significance of HLA class Il in breast | Guangzhou Medical |
| | | cancer | University, China |
| 7 | https://www.ncbi.nlm.nih.gov/ | Deregulation of HLA-I in cancer and | Ahmet Hazini, Oxford |
| | pmc/articles/PMC8344275/ | its central importance for | University, UK |
| | | immunotherapy | |
| 8 | https://www.ncbi.nlm.nih.gov/ | Role of cyclin D1 in | Fereshteh |
| | pmc/articles/PMC3908521/ | breast carcinoma | Mohammadizadeh et al., |
| | | | Babol University of |
| | | | Medical Sciences, |
| | | | Isfahan, Iran |
| 9 | https://www.frontiersin.org/ | Cancer Immune Evasion Through | Karthik |
| | articles/10.3389/ | Loss of MHC Class I Antigen | Dhatchinamoorthy et al., |
| | fimmu.2021.636568/full | Presentation | UMass Medical School, |
| | | | Worcester, MA |
| 10 | https://www.sciencedirect.com/ | Playing hide and seek: Tumor cells in | M.L.M. Jongsma et al., |
| | science/article/ | control of MHC class I antigen | Leiden University |
| | pii/S0161589021001607 | presentation | Medical Center, the |
| | | | Netherlands |
| 11 | https://portlandpress.com/ | Regulation of the antigen | Adithya |
| | biochemsoctrans/article/ | presentation machinery in cancer | Balasubramanian et al., |
| | 50/2/825/231082/ | and its implication for immune | The Walter and Eliza Hall |
| | Regulation-of-the- | surveillance | Institute of Medical |
| | antigen-presentation- | | Research, Melbourne, |
| | machinery | | Australia |
| 12 | https://www.frontiersin.org/ | The Immunology of Hormone | Jonathan Goldberg et al., |
| | articles/10.3389/ | Receptor Positive Breast Cancer | Dana-Farber Cancer |
| | fimmu.2021.674192/full | | Institute, Boston, MA |
| 13 | https://journals.plos.org/plosone/ | Identification of a Human Cyclin D1- | Tao Dao et al., MSKCC, |
| | article?id=10.1371/ | Derived Peptide that Induces Human | NY |
| | journal.pone.0006730 | Cytotoxic CD4 T Cells | |
| 14 | https://link.springer.com/ | HLA Alleles in Breast Cancer | Ozlem Goruroglu Ozturk, |
| | article/10.1007/s12253- | | Cukurova University, |
| | 012-9501-4 | | Adana, Turkey |
| 15 | https://pubmed.ncbi.nlm.nih.gov/ | HLA class-II allele frequencies in | Faik Deniz Gun et al., |
| | 21373933/ | Turkish breast cancer patients | Birecik State Hospital, |
| | | | Sanliurfa, Turkey |
| 16 | https://pubmed.ncbi.nlm.nih.gov/ | HLA-DRB1, -DQA1 and -DQB1 allele | Majid Mahmoodi et al., |
| | 21720852/ | and haplotype frequencies in female | Tehran University of |
| | | patients with early onset breast | Medical Sciences, Iran |
| | | cancer | |
| 17 | https://www.semanticscholar.org/reader/ | Negative association of the HLA- | Manar Fayiz Atoum et |
| | 7d9b68299bb8e99b02f18e8a68c256e36d91afa0 | DQB1*02 allele with breast cancer | al., Hashemite University, |
| | | development among Jordanians | Jordan |
| 18 | https://www.ejmanager.com/mnstemps/ | ANALYSIS OF HLA CLASS-I AND II | Ahmed A. Al-Hassan et |
| | 3/3-1430611055.pdf | ALLELES AND SERUM LEVELS OF | al., Al Nahrain University, |
| | | IL-2 AND IL-4 IN IRAQI | Iraq |
| | | WOMEN WITH BREAST CANCER | |
| 19 | https://journals.sagepub.com/doi/full/ | Immunogenetic Profiles and | Lisa M James et al., |
| | 10.1177/11769351221148588#bibr30- | Associations of Breast, Cervical, | Department of Veterans |
| | 11769351221148588 | Ovarian, and Uterine Cancers | Affairs Health Care |
| | | | System, Minneapolis, |
| | | | MN, USA |
| 20 | https://www.immunologyresearchjournal.com/ | Immunogenetic Association of 127 | Apostolos P. |
| | articles/immunogenetic- | Human Leukocyte Antigen (HLA) | Georgopoulos et al., |
| | association-of-127-human-leukocyte- | Alleles with 30 Cancers in | Department of Veterans |
| | antigen-hla-alleles-with-30- | Continental Western European | Affairs Health Care |
| | cancers-in-continental-western- | Countries | System, Minneapolis, |
| | european-countries.html | | MN, USA |
| 21 | https://www.ncbi.nlm.nih.gov/ | mRNA vaccination in breast cancer: | Xiao-ting Jiang et al., |
| | pmc/articles/PMC10132791 | current progress and future | Sun Yat-Sen University, |
| | | direction | Guangzhou, China |
| 22 | https://www.genome.jp/ | Breast Cancer Pathway | KEGG, Japan |
| | pathway/hsa05224 | | |
| 23 | https://www.genome.jp/ | Prostate Cancer Pathway | KEGG, Japan |
| | pathway/hsa05215 | | |
| 24 | https://www.nature.com/articles/ | Human CD4 cytotoxic T lymphocytes | Wen Lin et al., |
| | s42003-023-04812-3 | mediate potent tumor control in | Regeneron |
| | | humanized immune system mice | Pharmaceuticals, NY |
| 25 | https://www.frontiersin.org/ | CD4 CTL, a Cytotoxic Subset of | Arata Takeuchi et al., |
| | articles/10.3389/ | CD4+ T Cells, Their Differentiation | Niigata University, |
| | fimmu.2017.00194/full | and Function | Japan |
| 26 | https://www.frontiersin.org/ | The Era of Cytotoxic CD4 T Cells | Mara Cenerenti et al., |
| | articles/10.3389/ | | University of Geneva, |
| | fimmu.2022.867189/full | | Switzerland |
| 27 | https://www.bmbreports.org/journal/ | CD4+ cytotoxic T cells: an emerging | Seongmin Jeong et al., |
| | view.html?uid=1785&vmd=Full | effector arm of anti-tumor immunity | Daegu Gyeongbuk |
| | | | Institute of Science and |
| | | | Technology, South Korea |
| 28 | https://www.nature.com/ | Revisiting the role of CD4+ T cells in | Rong En Tay et al., |
| | articles/s41417-020-0183-x | cancer immunotherapy-new insights | Singapore Immunology |
| | | into old paradigms | Network, Singapore |
| 29 | https://www.ncbi.nlm.nih.gov/ | A novel vaccine for mantle cell | Jingtao Chen et al., Jilin |
| | pmc/articles/PMC4424584/ | lymphoma based on targeting cyclin | University, China; Baylor, |
| | | D1 to dendritic cells via CD40 | Dallas, TX; and more |
| | | | from the USA |

### BRIEF SUMMARY OF THE INVENTION

This invention is a cancer vaccine, i.e. it triggers an immune response to fight or prevent cancer after injecting the vaccine in the human body. It thereby supplements existing cancer treatments or makes some of them obsolete and/or makes the treatment more effective.

This invention utilizes that some cancers express MHC-II, and the antigens that these cancers over-express have been visible so far (prior to the cancer) only on MHC-I, where the T cell response has been reduced.

This invention utilizes the antigen's MHC-II, while it silences and thus avoids MHC-I, based on the personal allele composition. For the antigen Cyclin D1 the binding positions (i.e. the epitopes) are widely spread for different alleles, and this allows for a very short selection of epitopes into a polytope—that varies greatly with the personal allele composition. It is doable to select epitopes that bind to MHC-II while no part of them binds to MCH-I for most alleles when targeting Cyclin D1, because it expresses low on MHC-I (has few “strong binders” there). However the concept is generic for all antigens in all cancers that express MHC-II.

This invention trains immune system part MHC-II while it avoids harming healthy tissue and preventing as much as possible auto-immune disorders by not matching the immune system part MHC-I.

Cancers further often silence MHC-I, leaving side effects to healthy tissue unaffected in that the healthy tissue still expresses MHC-I.

The T cells of the immune system part MHC-II have generally not seen these epitopes before the cancer, because healthy tissue (except APCs) express only MHC-I.

The cancers that express MHC-II include:
Breast cancer (BC),
prostate cancer (PCa),
melanoma,
colorectal cancer,
ovarian cancer,
classic Hodgkin lymphoma,
glioma, and
non-small cell lung cancer

It may be that not all of MHC-II is expressed, in which case this must be taken into account.

This invention utilizes that CD4+ cells responding to MHC-II can be cytotoxic (CD4+ CTL), where this cytotoxic behavior is assumed to be reserved to MHC-I, to CD8+ CTLs. Or it utilizes a similar strong immune response, as suggested by references that point to the existence of protective MHC-II alleles.

Cyclin D1 has a low expression on healthy tissue, and Cyclin D1 is therefore likely to not be associated with inhibitory CD4+ Tregs (since at least outside the thymus they must come from the APCs).

The cancers that express both MHC-II and Cyclin D1 include:
Hormone positive breast cancer, (ER+ BC),
prostate cancer (PCa), and
melanoma.

The prediction of the binding to MHC-I and to MHC-II is calculated based on a computer algorithm taking the allele composition determined from a genomic test as input, from which the polytope that binds to MHC-II but not to MHC-I is derived. It may be that a “cut” or transition over two substrings in a polytope cannot avoid the creation of epitopes with a binding property on either MHC-I or on MHC-II no matter what amino acids are put in between the epitopes, then one of the substrings may either be discarded from the polytope, or the two separate polytopes may be devised as separate proteins in the vaccine.

The vaccine is then produced to include the polytope as an active ingredient (or several polytopes cf. above). The vaccine may as an alternative be selected from a set of pre-tested and approved set of vaccines with the desired behavior or most of it.

The vaccine may be a preventative vaccine (before the cancer is detected) or a treatment vaccine (after the cancer is detected). The vaccine may contain the polytope alone or together with other active ingredients or devised together with other vaccines. If the vaccine is given as several injections, it may be regulated, i.e. the dose of subsequent injections may be determined based on measures of effectiveness etc.

The vaccine may be one of the following vaccine technologies, or another:
mRNA vaccine,
SAM: Self Amplifying mRNA vaccine,
LNP mRNA: Lipid nanoparticles mRNA vaccine,
DC vaccine, or
ISV: In situ vaccine.

### Technical Field

The invention is in the area of vaccination, i.e. the injection of a chemical agent containing an active substance that invokes a reaction of the immune system in a human.

The presently disclosed subject matter relates generally to killing cells (i.e. inducing apoptosis by means of immune system cells attaching to cancer cells and other immune system responses).

The invention makes use of a computational environment in order to compute the right vaccine ingredient, which uses computational models that mimick the functioning of the immune system, including alleles of genes behind the immune system as well as the proteins that the system reacts on.

The invention further makes use of technologies to produce the vaccine based on the said computational model and the vaccine ingredient data. The invention does not comprise any of these further vaccine production technologies.

### DESCRIPTION

### Brief Description of the Drawings

FIG. 1: An overview of the human immune system with the Innate and the Adaptive parts, and where the Adaptive part is subdivided into B-cells and T-cells, and where T-cells are subdivided into CD4+—Helper T-cells—and CD8+—Cytotoxic or Killer T-cells.

FIG. 2: The two main types of T-cells (CD4+—Helper T-cells—and CD8+—Cytotoxic or Killer T-cells) differing by their co-receptor and both having a T Cell Receptor. Some T-cells are not shown.

FIG. 3: CD4+—T-cells bind to epitopes that are displayed by MHC-II, and CD8+—Cytotoxic or Killer T-cells—bind to epitopes that are displayed by MHC-I. In general MHC-II is only expressed on antigen presenting sells (APC) such as dendritic cells (DC) and the epitopes are in general from outside the human (e.g. extracellular bacteria), but some cancers express MHO-II and thereby present epitopes from proteins inside the cancer. This happens on MHC-I which all cells (with a nucleus) express, therefore also cancer cells. With the exception of APCs the MHC-I displays only epitopes from proteins inside the cell.

FIG. 4: The genes that are involved in determining the MHC-I and II respectively. Next to the gene is given the number of alleles and combinations.

FIG. 5: The CD4+ cells further subdivided into Th1, Th2, Th17, and Treg. The possible CTLs are not shown. The figure shows an APC expressing MHC-II, but it can also be some cancers.

FIG. 6: Cell types and MHC-I and II. The figure shows that some cancer cells (e.g. breast cancer, prostate cancer, melanoma, non-small cell lung cancer (NSCLC)) express (communicate on) MHO-II-something otherwise reserved for APCs, when there is no cancer. All cells with a nucleus communicate on MHC-I.

FIG. 7: The CD4+ cells having the role of (a) attracting CD8+ Cytotoxic T cells, (b) being cyctoxic themselves, and (c) helping B cells.

FIG. 8: “Cold” versus “Hot” Tumors showing MHC-II (MHC-I can also differentiate them, and is more used than MHC-II to differentiate). It is seen that the gene “CIITA” can upregulate the genes behind MHC-II and thus further the expression of MHO-II.

FIG. 9: The first step of the Antigen Presentation Pathway: Protein to MHC (I or II) binding. Part A: The protein e.g. “Cyclin D1” with its composition of amino acids, one per box. Part B: Modified protein to encompass only the strong binders. Part C: The same as part B, just emphasizing that there are a new set of amino acids that are now consecutive and will become epitopes, these epitopes should not become “strong binders” or “weak binders” and make unforeseen consequences of being presented—cf. part E. Part D: The modified protein of part C cut into epitopes by the phagocytosis of the cell (the length of 3 amino acids is shown as an example, in reality the epitopes are longer, often 15 for MHC-II). Part E: The epitopes that hold the new transitions of amino acids in the modified protein to be checked for binding to the MHC. Part F: The binding of an epitope to the MHC molecule—so that the epitope together with the MHC molecule can migrate to the surface of the cell and be displayed to the immune system (act as ligands to a T cell's receptor, TCR).

FIG. 10: Ranking of alleles both in MHC-I and MHC-II according to how well they are predicted to present the proteins shown in the header (Cyclin D1, Mucin 1, CEA, and TERT respectively)—these proteins being candidates as Antigens for Breast Cancer. It is seen that for Cyclin D1 in particular the alleles behind MHC-II all rank higher that the alleles behind MHC-I, whereas for the other proteins some of the alleles behind MHC-I (A*, B*, and C*) are ranked higher than some of the alleles behind MHC-II.

FIG. 11: The antigen staining of Cyclin D1 in different cancers—and to the right in normal tissue. In breast cancer, thyroid cancer, as well as prostate cancer and to some degree pancreatic cancer, the normal tissue staining is white (non-significant), and the cancer staining is elevated.

FIG. 12: The antigen staining of Mucin 1 in different cancers—and to the right in normal tissue. In the normal tissue of breast and prostate cancers the expression is elevated.

FIG. 13: The antigen staining of CEA in different cancers—and to the right in normal tissue.

FIG. 14: The expression of Cyclin D1 in different normal tissue. The maximum is 120 nTPM (compare to Mucin 1 and CEA)

FIG. 15: The expression of Mucin 1 in different normal tissue. The maximum is 1,000 nTPM.

FIG. 16: The expression of CEA in different normal tissue. The maximum is 1,000 nTPM.

FIG. 17: The Breast Cancer Pathway in KEGG. It can be seen (highlighted by the box to the right) that ER+ (Estrogen Receptor positive) breast cancers produce Cyclin D1 (with the gene abbreviation CCND1) (among others).

FIG. 18: The Prostate Cancer Pathway in KEGG. It can be seen (highlighted by the box at the bottom right) that some of these cancers produce Cyclin D1 (among others).

FIG. 19: The Melanoma Pathway in KEGG. It can be seen (highlighted by the box at the bottom right) that some of these cancers produce Cyclin D1 (among others).

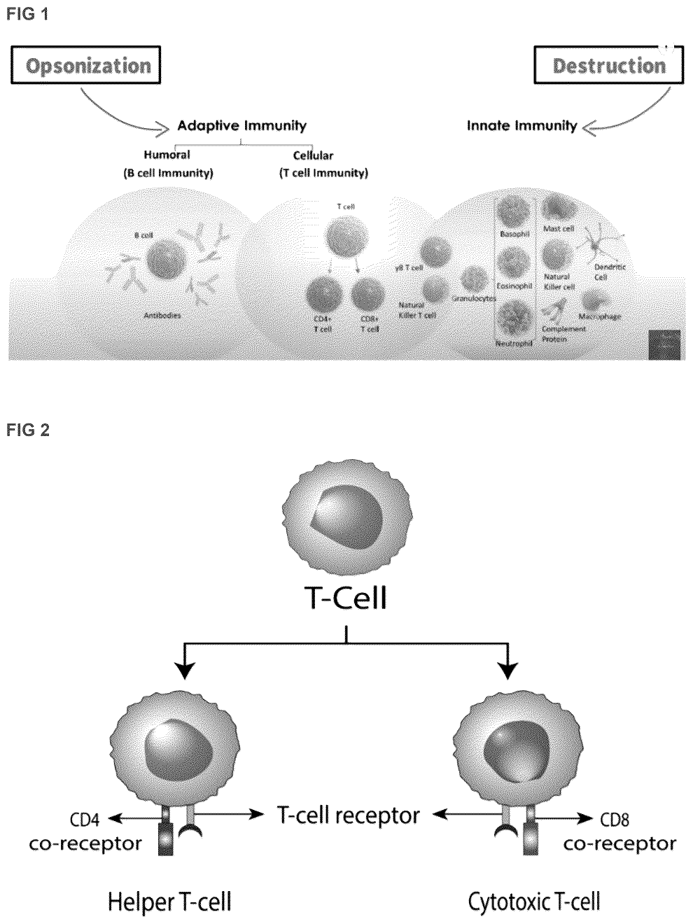

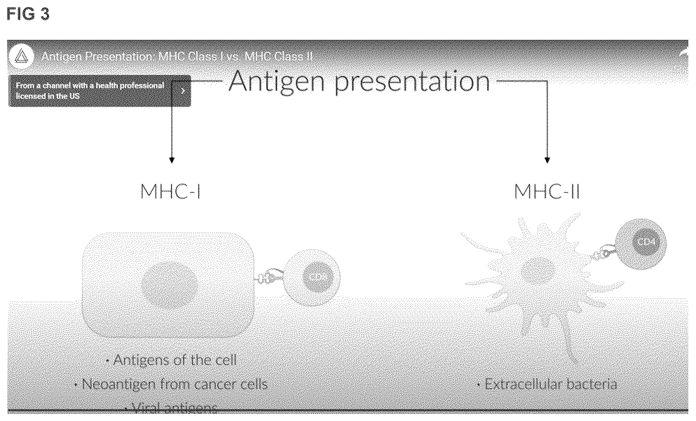

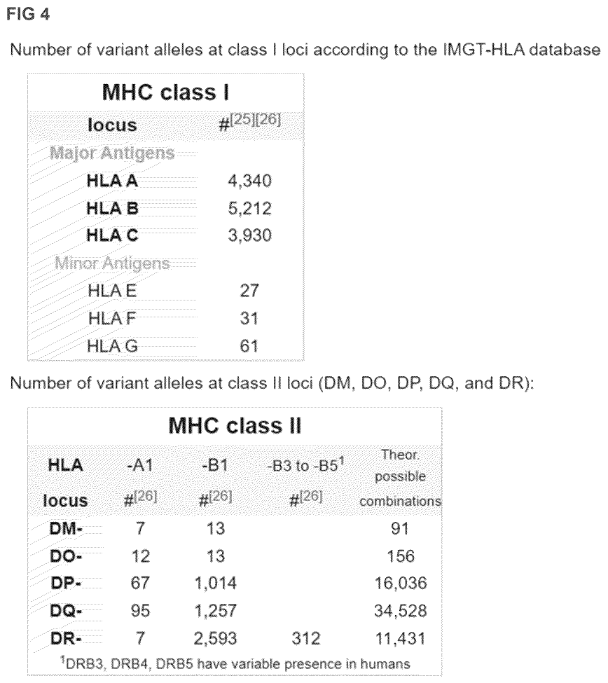

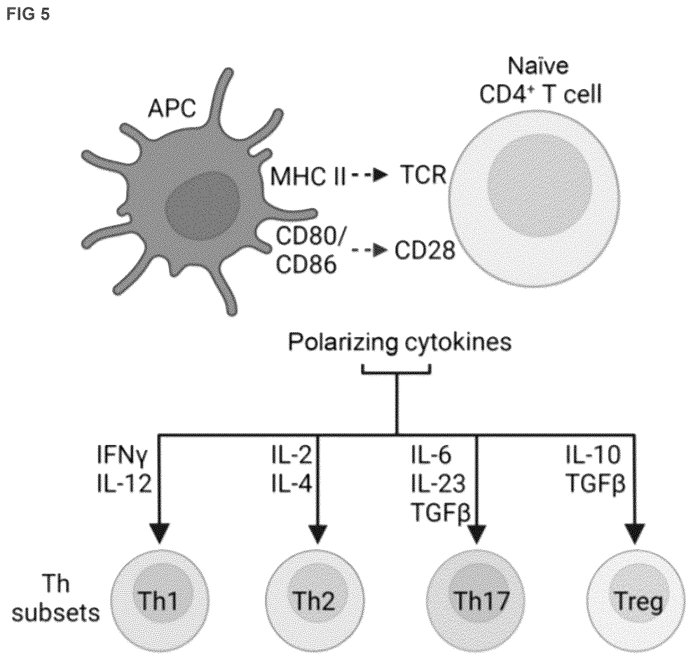

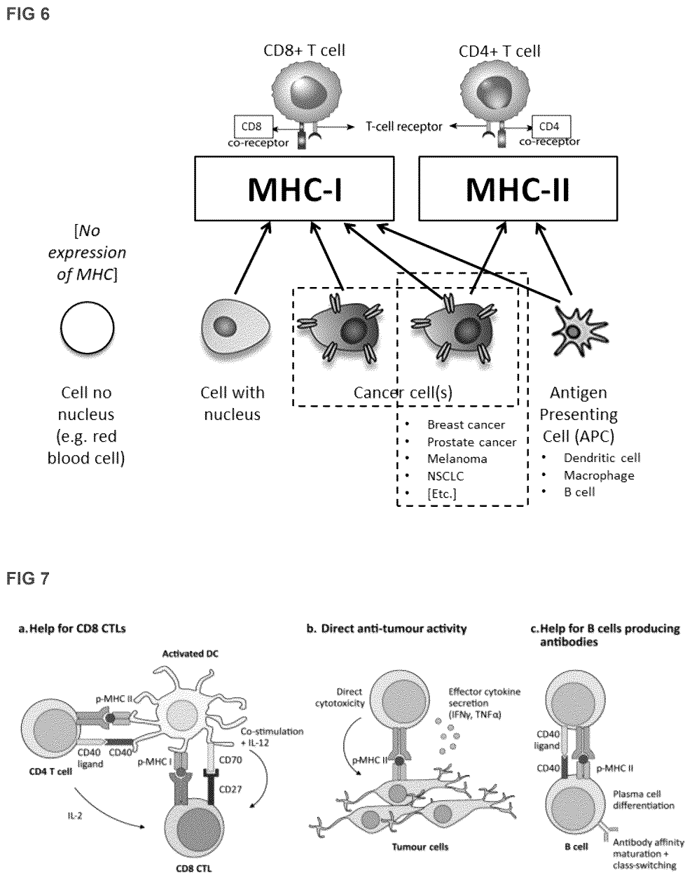

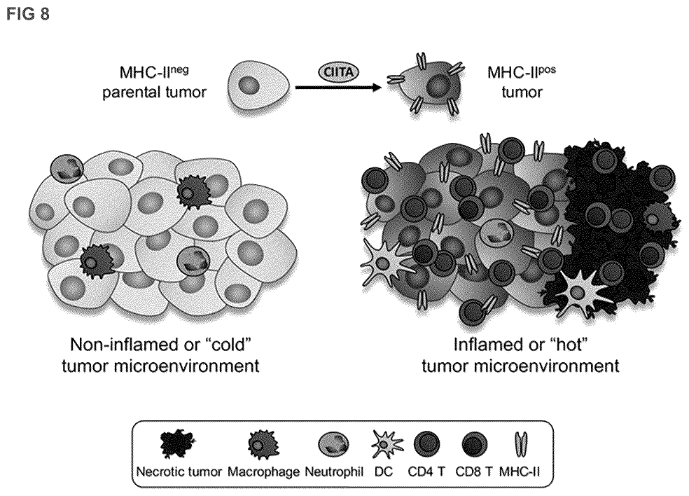

### DETAILED DESCRIPTION OF THE INVENTION

The invention relates to triggering a response from the immune system by injecting once or several times an agent containing an antigen (a protein) or parts of the antigen, a so-called polytope, or several polytopes. The purpose of the immune response is to partly or fully eradicate the cells that represent the illness, which may be cancer, in the following “the cancer”.

If a test is carried out on the human, and if it shows that the cancer expresses MHC-II or part of it as well as the said Antigen, the said Polytope can possibly be designed such that it binds to and therefore possibly displays on MHC-II and not on MHC-I. It therefore elicits an immune response that may lead to fully or partly eradicating the cancer without implying the cells that represent the healthy tissue and express MHC-I. Except for those humans that have a cancer expressing MHC-II, but where the antigen doesn't bind to MHC-II and therefore does not display. For Cyclin D1 it is estimated that 0.3% of humans having no strong binder for MHC-II.

The Antigen protein that we consider the most appropriate for this is Cyclin D1 (having the gene code CCND1). It has a low expression on MHC-I, and it is expressed with a low level in healthy tissue cells. It is likely that the immune system has not prior to the vaccination produced any T cells for MHC-II with T cell receptors (TCR) that match a polytope based on Cyclin D1, because prior to the cancer only the low level of Cyclin D1 in APCs has displayed on MHC-II.

The trick is then to select a subset of the Cyclin D1 protein for vaccination that is completely or almost silent on MHC-I but visible on MHC-II. Then there will be no added CD8+ CTLs, and the added CD4+ cytotoxic T cells and other immune response on MHC-II will only kill cancers and maybe a few additional APCs.

If the invention is used in the treatment setting, the invention further involves making a test on a sample from the cancer (e.g. a biopsy) or other expert knowledge to establish whether (1) the cancer expresses MHC-II or a part of it and (2) the cancer over-expresses the Antigen (e.g. Cyclin D1), i.e. significantly more than it is expressed in healthy tissue. It is expected that a subset of Breast Cancer, Prostate Cancer, and Melanoma all express both MHC-II ond over-express Cyclin D1.

A genomic test is carried out in order to derive the allele composition of the HLA genes of the human (e.g. a swab of the mouth). This test includes a subset of or the complete set of the alleles of the HLA genes:
MHC-I: HLA-A, HLA-B, HLA-C, HLA-E, HLA-F, HLA-G, and HLA-H.
MHC-II: HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, and HLA-DRB5.

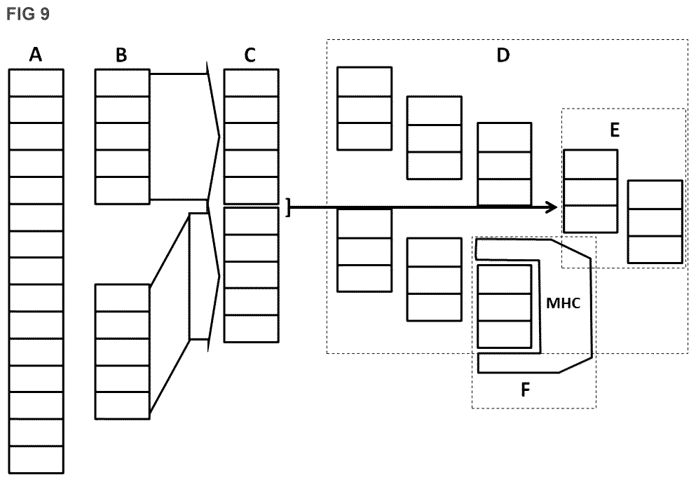

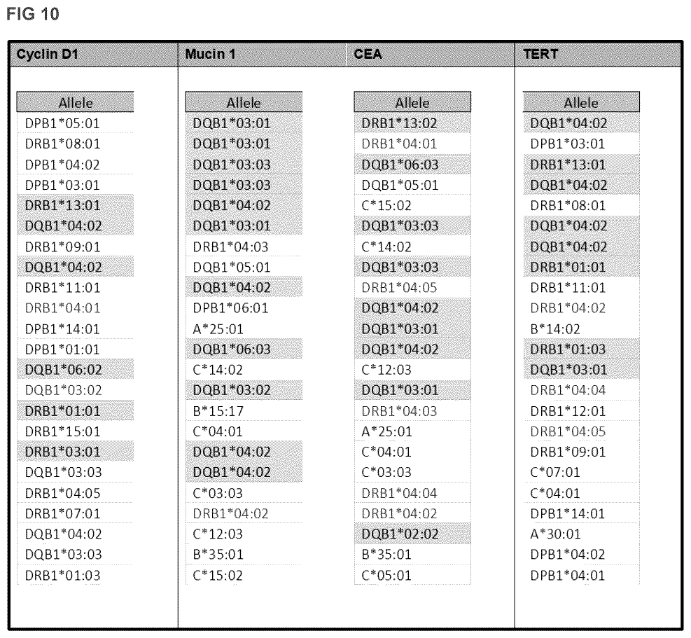

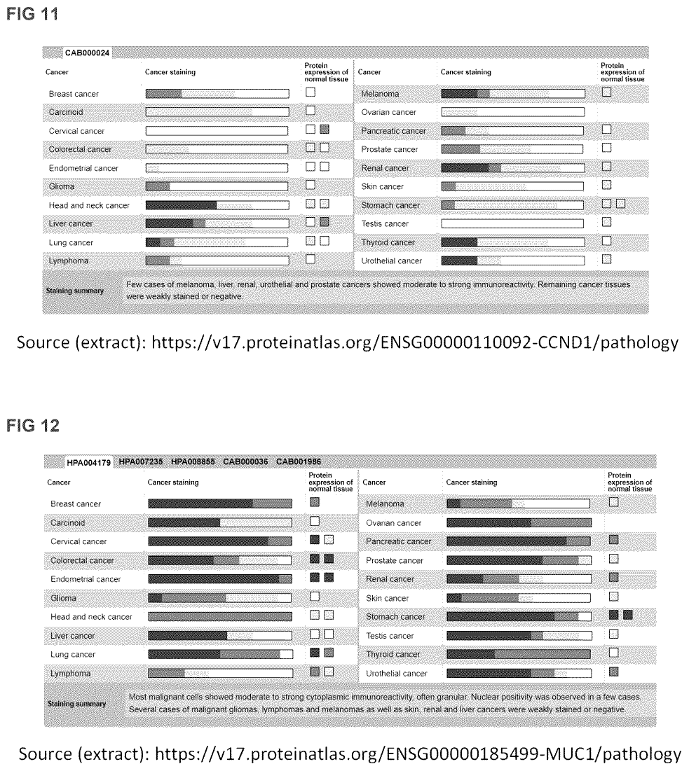

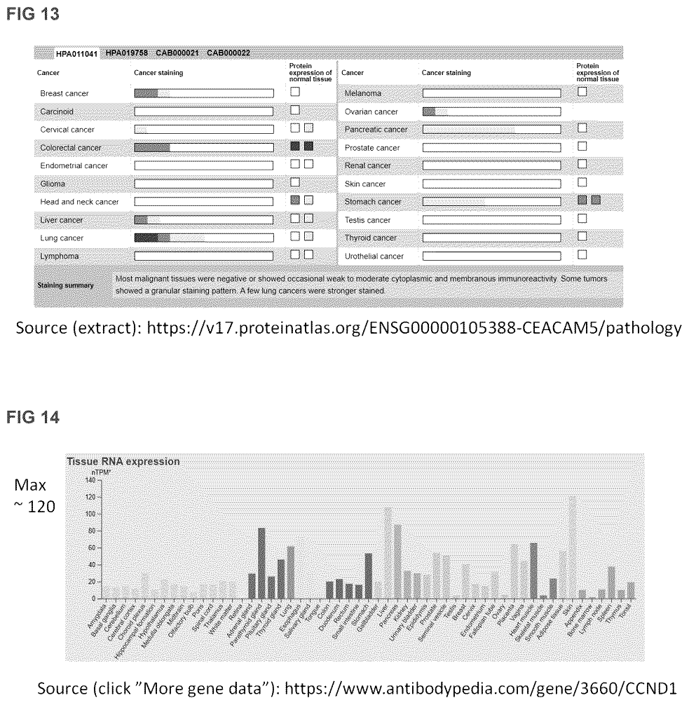

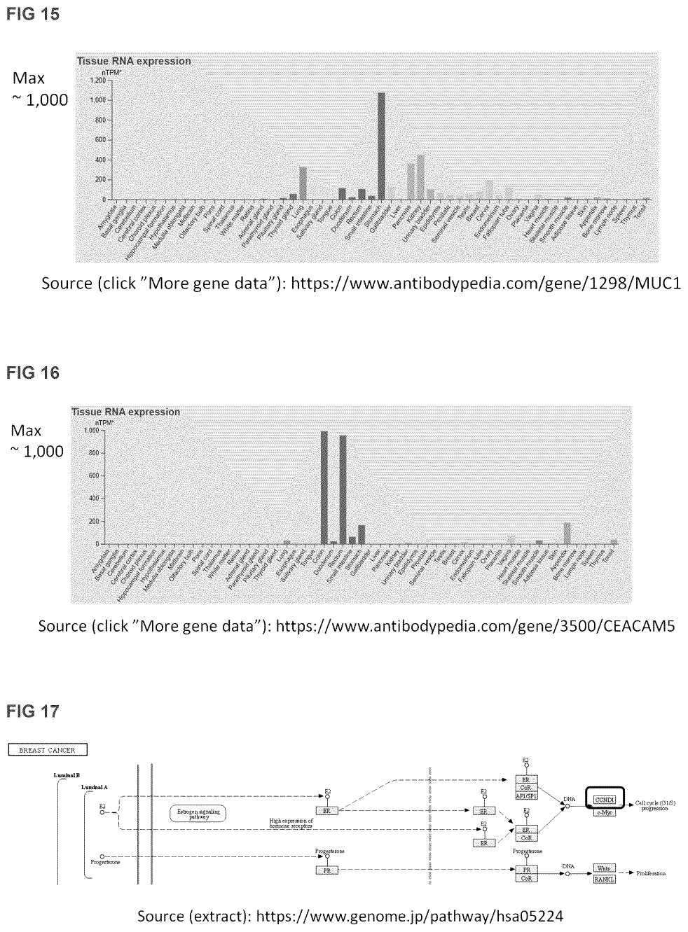

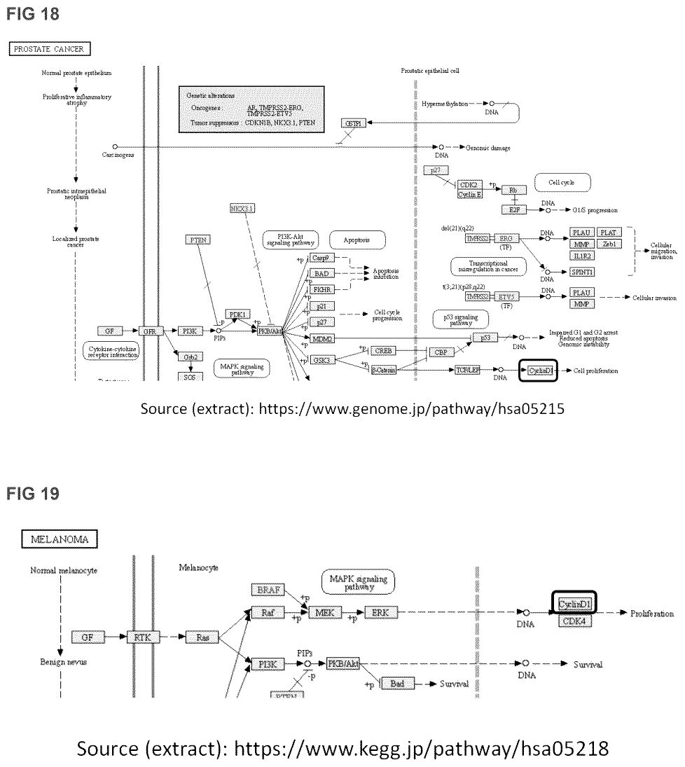

A computer program as currently defined in the services [S4] and [S5] is used to predict the binding data for the antigen (e.g. Cyclin D1 and subsequently for polytopes derived from it cf. the description below) given particular alleles or haplotypes (combinations of alleles). The computer program provides as part of its output a property for each epitope and each allele being one of: a “non-binder”, a “weak binder” (WB), and a “strong binder” (SB), where the definitions of WB and SB are different for MHC-I and II, but they mean the same: They represent the likelihood of the epitope binding to that MHC molecule (given the allele): A “strong binder” (SB) is likely to bind to the MHC in question cf. F on FIG. 9 and can thus show together with the MHC molecule on the cell surface to the immune system, a “weak binder” (WB) may or may not bind to the MHC and show together with the MHC molecule on the cell surface to the immune system, with a likelihood of more than 1% but less than 100%, and a “non-binder” will not bind to the MHC and therefore with almost certainty not show together with the MHC molecule on the cell surface to the immune system.

Using an antigen polytope as a vaccination agent to trigger an immune reaction on MHC-II of the adaptive immune system, either as prevention of, or as treatment of, a cancer of the types that express MHC-II. The antigen polytope vaccine may be together with other agents in the vaccine that trigger other immune responses on MHC-I or MHC-II or both.

The said Polytope is determined by the following steps 1 to 6:

### Step 1:

We construct, using the output from the service of [S5] or the stand alone service or a similar or optimized service for MHC-II, a database with or list of the binding properties of the combination of
all epitopes of the antigen on MHC-II (epitopes of the length 13-25 amino acids, or a subset of these epitopes having lengths e.g. just one length 15, out of the full length of the antigen cf. A on FIG. 9) and.
all alleles (or haplotypes) constituting the different MHC-II molecules.
This implies identifying an epitope as one of (1) a non-binder, (2) a “weak binder” (WB), or (3) a “strong binder” (SB)—with any particular allele or combination af alleles.

### Step 2:

Selection of those epitopes (by e.g. position in the sequence of the full length antigen) that constitute “strong binders” (SBs) with the specific combination of alleles for that person. Cf. B on FIG. 9. The “strong binders” may constitute sequences that are consecutive epitopes that are longer than the length of the individual epitopes—in FIG. 9 the epitope length of 3 is used as a simplistic example cf. D on FIG. 9. If there are no “strong binders” for the specific allele combination, then include the WBs.

### Step 3:

Construction of an “a priori polytype” by appending the epitopes above-marking the “cuts” i.e. the joining points of epitopes in the polytope. The said Cuts are all inside the polytope. Cf. C on FIG. 9. The said A Priori Polytope is then modified as follows:

### Step 4:

Check on both MHC-I and MHC-II, using the services of [S4] and [S5] or the stand alone services or similar or optimized services, whether the said A Priori Polytope, focusing on the epitopes that are now created around the said Cuts, will trigger a “strong binder” (SB) or a “weak binder” (WB) on either of MHC-I or MHC-II. Cf. E on FIG. 9. This will constitute a biological behavior that is unprecedented or unusual and not tested. Although the figure displays a usual epitope length (of 3 in the figure), this check needs to be performed for all possible epitope lengths, i.e. 8 to 14 on MHC-I and 13 to 25 on MHC-II.

### Step 5:

Padding the Cuts that introduce a strong or weak binder on either MHC-I or MHC-II or both: (1) If the “Cut” is wide (it the positions are far apart) then any sequence that either, (1A) silences the Cut (makes it a non-binder) or (1B) extends the epitope to include more from the original protein (padding with the amino acids of the original string) until the cut is silenced (it is a non-binder). (2) If the “Cut” is close (the positions are close) then the original string of the full protein must be used so that the epitopes join up to a consecutive substring of the protein and thereby is similar to what comes out of the full length protein. Cf. the progression between A, B, and C on FIG. 9, where more parts of A are added to create longer substrings in B and “pad” the gap when joined in C.
If the said A Priori Polytope after said Padding removes all WB or SB from both MHC-I and MHC-II, the said A Priori Polytope is updated to include the said Padding.
If it cannot be avoided that a substring in the said A Priori Polytope after the said Padding creates a WB or SB in either MHC-I or MHC-II or both, this substring is taken out of the A Priori Polytope, i.e. the said A Priori Polytope is updated to exclude the substring. In this case, we go back to and repeat Step 4, i.e. we need to check the A Priori Polytope again on both MHC-I and on MHC-II.
It is considered to use the substring thus taken out as a separate Polytope and thus as a separate protein in the vaccination, provided it passes the subsequent Step 6.

### Step 6:

If there are “strong binders” on MHC-I that have been selected for the A Priori Polytope (these SBs on MHC-I will most likely be associated with epitopes embedded in the SB epitopes on MHC-II, since the length of epitopes is shorter in most cases on MHC-I than on MHC-II, and we have changed or removed those SB epitopes that come from “Cuts” in the steps above), these epitopes on MHC-II must be thrown out, i.e. the A Priori Polytope is updated to not include the epitopes that constitute SBs on MHC-II, if the said Epitopes contain (or are associated with) epitopes that are SBs on MHC-I.
If this introduces a new cut, repeat Step 4, i.e. we need to check the A Priori Polytope again on both MHC-I and on MHC-II.
If this results in no epitopes left in the polytope then either (1) leave them in (and accept the side effect of it triggering an immune response on MHC-I) or (2) include the “weak binders” on MHC-II. We prefer (1) since there are likely no TCR that match anyhow.

The vaccine is manufactured using the Polytope sequence as the active ingredient, converted to mRNA. It is administered (typically injected) such that the human body will generate a protein similar to the Polytope in e.g. an APC that expresses MHC-II and thus make the immune system react to this protein by e.g. generating CD4+ T cells that can also attach to the cancer and kill it.

The vaccine may need to be repeated in order to create the desired effect (of either prevention or treatment).

The effect of the vaccine may be monitored and assessed to see if (in case of prevention) CD4+ CTLs or antibodies are produced or (in case of treatment) the cancer cells are furthermore being killed by the immune system. The dose of the vaccination may be adjusted according to this monitoring or assessment.

### BEST MODE FOR CARRYING OUT THE INVENTION

We describe the use of the antigen Cyclin D1 below in a vaccine. Using a Cyclin D1 polytope as a vaccination agent to trigger an immune reaction on MHC-II of the adaptive immune system, either as prevention of, or as treatment of, a cancer of the types either (1) breast cancer, (2) prostate cancer, or (3) melanoma.

The said Polytope is determined by the following steps, which are a special case of the aforementioned Steps 1-6—which Steps are referenced and detailed for implementation with Cyclin D1 in the following:

### Step 1:

A database table is constructed containing the binding properties of all epitopes of Cyclin D1 defined in the sequence number 1 named “Cyclin D1” on MHC-II (epitopes of the length 15 amino acids, varying from 13 to 25, out of the full length of Cyclin D1, which is 295) on all alleles (or haplotypes) of MHC-II. This includes identifying any epitope as one of (1) a non-binder, (2) a “weak binder” (WB), or (3) a “strong binder” (SB).

Across all epitopes with the length of 15, using the service [S5] with a selection of alleles that constitute most of the world's population according to the frequencies on the service [S7], we get the following count of “strong binders”:

| Pos | Epitope (of length 15) | Count of SB |
|---|---|---|
| 10 | VETIRRAYPDANLLN | 11 |
| 11 | ETIRRAYPDANLLND | 28 |
| 12 | TIRRAYPDANLLNDR | 41 |
| 13 | IRRAYPDANLLNDRV | 11 |
| 18 | PDANLLNDRVLRAML | 1 |
| 19 | DANLLNDRVLRAMLK | 2 |
| 22 | LLNDRVLRAMLKAEE | 2 |
| 23 | LNDRVLRAMLKAEET | 5 |
| 24 | NDRVLRAMLKAEETC | 2 |
| 25 | DRVLRAMLKAEETCA | 2 |
| 47 | CVQKEVLPSMRKIVA | 3 |
| 48 | VQKEVLPSMRKIVAT | 3 |
| 49 | QKEVLPSMRKIVATW | 3 |
| 55 | SMRKIVATWMLEVCE | 3 |
| 56 | MRKIVATWMLEVCEE | 11 |
| 57 | RKIVATWMLEVCEEQ | 2 |
| 79 | PLAMNYLDRFLSLEP | 2 |
| 84 | YLDRFLSLEPVKKSR | 1 |
| 85 | LDRFLSLEPVKKSRL | 2 |
| 86 | DRFLSLEPVKKSRLQ | 5 |
| 87 | RFLSLEPVKKSRLQL | 3 |
| 88 | FLSLEPVKKSRLQLL | 2 |
| 104 | ATCMFVASKMKETIP | 12 |
| 105 | TCMFVASKMKETIPL | 11 |
| 108 | FVASKMKETIPLTAE | 2 |
| 109 | VASKMKETIPLTAEK | 16 |
| 110 | ASKMKETIPLTAEKL | 19 |
| 111 | SKMKETIPLTAEKLC | 9 |
| 112 | KMKETIPLTAEKLCI | 6 |
| 116 | TIPLTAEKLCIYTDN | 2 |
| 119 | LTAEKLCIYTDNSIR | 6 |
| 120 | TAEKLCIYTDNSIRP | 8 |
| 121 | AEKLCIYTDNSIRPE | 20 |
| 122 | EKLCIYTDNSIRPEE | 6 |
| 124 | LCIYTDNSIRPEELL | 7 |
| 125 | CIYTDNSIRPEELLQ | 10 |
| 126 | IYTDNSIRPEELLQM | 7 |
| 127 | YTDNSIRPEELLQME | 17 |
| 128 | TDNSIRPEELLQMEL | 20 |
| 129 | DNSIRPEELLQMELL | 14 |
| 133 | RPEELLQMELLLVNK | 4 |
| 134 | PEELLQMELLLVNKL | 3 |
| 135 | EELLQMELLLVNKLK | 17 |
| 144 | LVNKLKWNLAAMTPH | 6 |
| 145 | VNKLKWNLAAMTPHD | 16 |
| 146 | NKLKWNLAAMTPHDF | 1 |
| 148 | LKWNLAAMTPHDFIE | 2 |
| 149 | KWNLAAMTPHDFIEH | 2 |
| 152 | LAAMTPHDFIEHFLS | 4 |
| 153 | AAMTPHDFIEHFLSK | 13 |
| 154 | AMTPHDFIEHFLSKM | 13 |
| 155 | MTPHDFIEHFLSKMP | 4 |
| 159 | DFIEHFLSKMPEAEE | 2 |
| 160 | FIEHFLSKMPEAEEN | 3 |
| 161 | IEHFLSKMPEAEENK | 7 |
| 162 | EHFLSKMPEAEENKQ | 2 |
| 173 | ENKQIIRKHAQTFVA | 1 |
| 188 | LCATDVKFISNPPSM | 5 |
| 189 | CATDVKFISNPPSMV | 16 |
| 190 | ATDVKFISNPPSMVA | 40 |
| 191 | TDVKFISNPPSMVAA | 17 |
| 192 | DVKFISNPPSMVAAG | 6 |
| 193 | VKFISNPPSMVAAGS | 1 |
| 198 | NPPSMVAAGSVVAAV | 29 |
| 199 | PPSMVAAGSVVAAVQ | 63 |
| 200 | PSMVAAGSVVAAVQG | 79 |
| 201 | SMVAAGSVVAAVQGL | 42 |
| 202 | MVAAGSVVAAVQGLN | 18 |
| 205 | AGSVVAAVQGLNLRS | 1 |
| 206 | GSVVAAVQGLNLRSP | 2 |
| 214 | GLNLRSPNNFLSYYR | 2 |
| 218 | RSPNNFLSYYRLTRF | 1 |
| 219 | SPNNFLSYYRLTRFL | 1 |
| 220 | PNNFLSYYRLTRFLS | 1 |
| 247 | CQEQIEALLESSLRQ | 2 |
| 248 | QEQIEALLESSLRQA | 6 |
| 249 | EQIEALLESSLRQAQ | 1 |
| 256 | ESSLRQAQQNMDPKA | 1 |
| 262 | AQQNMDPKAAEEEEE | 1 |
| 263 | QQNMDPKAAEEEEEE | 9 |
| 264 | QNMDPKAAEEEEEEE | 15 |
| 265 | NMDPKAAEEEEEEEE | 38 |
| 266 | MDPKAAEEEEEEEEE | 19 |
| 267 | DPKAAEEEEEEEEEV | 8 |

According to Step 1 we narrow that down based on a particular combination of MHC-II alleles, e.g. the combination DPA1_0202+DPB1_0501+DQA1_0501+DQB1_0301+DRB1_0701, which will give us 8 “strong binders” cf. Step 3 below. We can see from the above list, that the result of narrowing it down will be very different for different allele combinations.

We estimate that about 0.3% of the world's population will have an allele combination on MHC-II that gives no “strong binders” for Cyclin D1 on MHC-II.

### Step 2:

Selection of those epitopes (by e.g. position in the sequence of the full length Cyclin D1 protein of 295 defined in the sequence named “Cyclin D1”) that constitute “strong binders” (SBs) with the specific combination of alleles for that person. The “strong binders” may constitute substrings that are consecutive epitopes that together are longer than the length of the individual epitopes.
If there are no “strong binders” for the specific allele combination, something which will occur in approx. 0.3% of cases, then we include the WBs in this selection.

### Step 3:

Construction of an “a priori polytype” by appending the epitopes above-marking the “cuts” i.e. the joining points of epitopes in the polytope.

For the allele/haplotype combination DPA1_0202+DPB1_0501+DQA1_0501+DQB1_0301+DRB1_0701 we get the sequence number 3 named “Cyclin D1 a priori polytope DPA1_0202_DPB1_0501_DQA1_0501_DQB1_0301_DRB1_0701” or

| Pos | Epitope | Distance | Append |
|---|---|---|---|
| 85 | LDRFLSLEPVKKSRL | | LDRFLSLEPVKKSRL |
| 86 | DRFLSLEPVKKSRLQL | 1 | QL |
| 197 | SNPPSMVAAGSVVAAV | 111 | SNPPSMVAAGSVVAAV |
| 199 | PPSMVAAGSVVAAVQ | 2 | Q |
| 200 | PSMVAAGSVVAAVQG | 1 | G |
| 201 | SMVAAGSVVAAVQGL | 1 | L |
| 202 | MVAAGSVVAAVQGLN | 1 | N |
| 206 | GSVVAAVQGLNLRSP | 4 | LRSP |

Where the nuclear acids in the column “Append” make up the sequence of the A Priori Polytope. The polytope above has one Cut, positioned between the epitope that begins in the Position (Pos) 86 and epitope that begins in the Position 197.

### Step 4:

Check on both MHC-I and MHC-II whether the said A Priori Polytope, focusing on the epitopes that are now created around the Cuts, will trigger a “strong binder” (SB) or a “weak binder” (WB) on either of MHC-I or MHC-II. This check needs to be performed for all possible epitope lengths, i.e. 8 to 14 on MHC-I and 13 to 25 on MHC-II.

### Step 5:

Padding the cuts that introduce a strong or weak binder on either MHC-I or MHC-II or both: (1) If the “Cut” is wide (if the positions are far apart) then any sequence that either, (1A) silences the cut (makes it a non-binder) or (1B) extends the epitope to include more from the original protein (padding with the amino acids of the original string) until the cut is silenced (it is a non-binder). (2) If the Cut is close (the positions are close) then the original string of the full protein must be used so that the epitopes join up to a consecutive substring of the protein and thereby is similar to what comes out of the full length protein. Cf. the progression between A, B, and C on FIG. 9, where more parts of A are added to create longer substrings in B and “pad” the gap when joined in C.
If the said A Priori Polytope after said Padding removes all WB or SB from both MHC-I and MHC-II, the said A Priori Polytope is updated to include the said Padding.
If it cannot be avoided that a substring in the said A Priori Polytope after the said Padding creates a WB or SB in either MHC-I or MHC-II or both, this substring is taken out of the A Priori Polytope, i.e. the said A Priori Polytope is updated to exclude the substring. In this case, we go back to and repeat Step 4, i.e. we need to check the A Priori Polytope again on both MHC-I and on MHC-II.

### Step 6:

If there are “strong binders” on MHC-I that have been selected for the A Priori Polytype, the A Priori Polytope is updated to not include the epitopes that constitute SBs on MHC-II, if the said Epitopes contain (or are associated with) epitopes that are SBs on MHC-I.

Then to follow the example above the allele/haplotype combination on MHC-II DPA1_0202+DPB1_0501+DQA1_0501+DQB1_0301+DRB1_0701 and for the allele combination on MHC-I A_0201+B_0702+C_0701 we get the sequence number 2 named “Cyclin D1 polytope

A_0201_B_0702_C_0701_DPA1_0202_DPB1_0501_DQA1_0501_DQB1_0301_DR B1_0701” or

| Pos | Epitope | Distance | Append |
|---|---|---|---|
| 197 | SNPPSMVAAGSVVAAV | 111 | SNPPSMVAAGSVVAAV |
| 199 | PPSMVAAGSVVAAVQ | 2 | Q |
| 200 | PSMVAAGSVVAAVQG | 1 | G |
| 201 | SMVAAGSVVAAVQGL | 1 | L |
| 202 | MVAAGSVVAAVQGLN | 1 | N |

Where the nuclear acids in the column “Append” make up the sequence of the A Priori Polytope.
If this introduces a new cut, repeat Step 4, i.e. we need to check the A Priori Polytope again on both MHC-I and on MHC-II.
If this results in no epitopes left in the polytope then leave them in (and accept the side effect of it triggering an immune response on MHC-I)

The vaccine is manufactured using the above Polytope sequence as the active ingredient, converted to mRNA. It can be done as an mRNA vaccine or a SAM vaccine that makes dendritic cells (“DC”-a subset of the APCs) produce a protein similar to the above Polytope, such that the MHC-II expressed on the said DCs will activate the immune system e.g. by making it produce CD4+ T cells that can also react on the cancer cells.

It may be implemented such that a reduced set of vaccines will meet the requirements of most of the world's population-without meeting the optimal combination of SBs on MHC-II for a particular allele combination, but not being excessive in length outside the SBs on MHC-II, and not invoking any WBs or SBs on either of MHC-I or MHC-II from the Cuts, and not invoking any SBs on MHC-I. This will mean that we can pre-approve those vaccines having specified sequences together with their allele combinations instead of relying on an approval of the above method of Step 1-6.

### SEQUENCE LISTING XML

A sequence listing XML submitted as an xml file is incorporated herein by reference. The sequence listing XML file submitted has the name “Cyclin D1 Based Cancer Vaccine.xml”, was created on Apr. 5, 2024, and is 3,966 bytes in size.

1. A method for generating an active ingredient consisting of one or several proteins in a vaccine that prevents or treats human cancers that express MHC-II in full or in part—referred to as “MHC-II” in the following—on the cell surface by
(1) activating the MHC-II part of the adaptive immune system to discover the cancer by maximizing the likelihood of displaying, as a consequence of injecting the said Vaccine, some of a first set of epitopes, that when concatenated (without repeating the amino acids that make up the said Epitopes), are similar to the said Active Ingredient,
(2) so that the displayed epitopes of said First Set of Epitopes are also substrings of an antigen produced by the said Cancer, which said Epitopes the said Cancer may display on MHC-II, and
(3) while not activating the MHC-I part of the adaptive immune system by maximizing the likelihood that no part of the said Active Ingredient when deriving any second set of epitopes, that when concatenated (without repeating the amino acids that make up the said Epitopes) make up the said Active Ingredient, will display on MHC-I, thereby minimizing any undesired side effects in the human as well as minimizing the likelihood of autoimmune disorders as a consequence of injecting the said Vaccine,
(4) while maximizing the likelihood that no element in a third set of epitopes, of either the said First Set of Epitopes or Second Set of Epitopes, that are not parts of said Antigen (because the epitopes of said Third Set of Epitopes span joins or cuts in the said Antigen, so that the said Epitopes do not exist in the said Antigen) will display on MHC-I nor on MHC-II,
(5) basing the specification of the said Active Ingredient on an algorithm that predicts the behavior of the immune system for the said Antigen as well as for any sequence of epitopes that are considered to make up the said Active Ingredient, given data on the gene alleles of the said Human, in terms of computing a binding probability for each epitope of the said First Set of Epitopes for it to bind to MHC-II and in terms of computing a binding probability for each epitope of the said Second Set of Epitopes for it to bind to MHC-I.

2. A method according to claim 1 where the said Cancer that expresses MHC-II in full or in part on the cell surface is one of
Breast cancer (BC),
prostate cancer (PCa),
melanoma,
colorectal cancer,
ovarian cancer,
classic Hodgkin lymphoma,
glioma, and
non-small cell lung cancer.

3. A method according to claim 1 where the said Antigen besides being produced by the said Cancer is also present in some of the non-cancerous cells of the said human, which Non-Cancerous Cells except for Antigen Presenting Cells (APCs) do not express MHC-II, and the said Antigen is therefore a Tumor Associated Antigen.

4. A method according to claim 1 where the said Active Ingredient is one protein, said Protein called a Polytope.

5. A method according to claim 1 where the epitopes in the said First Set of Epitopes have a length (number of amino acids) of one of 13, 14, 15, 16, 17, 18, 19, 20, 21, 22, 23, 24, and 25 amino acids, and the epitopes in the said Second Set of Epitopes have a length (number of amino acids) of one of 8, 9, 10, 11, 12, 13, and 14 amino acids.

6. A method according to claim 1 where the said Algorithm classifies the said Binding Probabilities for an epitope to bind to MHC-I as well as to MHC-II into one of (1) a non-binder, (2) a weak binder (WB), and a strong binder (SB), such that “maximizing the likelihood of an epitope of the said First Set of Epitopes to bind to MHC-II” is implemented by selecting the said Epitope as an SB on MHC-II, and such that “maximizing the likelihood of an epitope of the said Second Set of Epitopes not to bind to MHC-I” is implemented by selecting the said Epitope as one of (1) a Non-Binder and (2) a WB on MHC-I, and such that “maximizing the likelihood of an epitope of the said Third Set of Epitopes not to display on MHC-I nor on MHC-II” is implemented by selecting the said Epitope as a Non-Binder on both MHC-I and on MHC-II.

7. A method according to claim 1 where said Algorithm is implemented as the combination of the services in [S4], currently accessed on the URL “https://services.healthtech.dtu.dk/services/NetMHCpan-4.1/”, or as a subsequently improved version of [S4], for MHC-I, and as in [S5], currently accessed on the URL “https://services.healthtech.dtu.dk/services/NetMHCIIpan-4.3/”, or as a subsequently improved version of [S5], for MHC-II, modified to take into account of the situation that it may be that not all of MHC-II is expressed by the said Cancer.

8. A method according to claim 6 where, if after selecting among the said First Set of Epitopes to bind to MHC-II as an SB has as a consequence that the selection is empty (if there are no SB on MHC-II), then “maximizing the likelihood of an epitope of the said First Set of Epitopes to bind to MHC-II” is implemented by selecting the said Epitope as a WB on MHC-II.

9. A method according to claim 6 where, if after selecting among the said Second Set of Epitopes to bind to MHC-I as an SB has as a consequence that the said Active Ingredient is empty (if there are no binders left for MHC-II after removing the SB of MHC-I), then it is implemented to not enforce the requirement to identify any said Second Set of Epitopes and then flag this situation (allowing the method to go on without removing the epitopes that may bind to MHC-I, since it is likely that there are a limited number of CD8+ T cells that bind to these epitopes in MHC-I due to the T cell selection, but with a warning that unwanted side effects or auto-immune disorders may occur).

10. A method according to claim 4 where the said Polytope consists of only consecutive epitopes with the Third Set of Epitopes is empty (so that there are no “cuts” in the said Polytope).

11. A method according to claim 3 where the said Tumor Associated Antigen is Cyclin D1 with the gene code CCND1 and as defined in the sequence number 1 named “Cyclin D1”.

12. A method according to claim 1 where the said Vaccine is produced and administered to the body of the human as one of
mRNA vaccine,
SAM: Self Amplifying mRNA vaccine,
LNP mRNA: Lipid nanoparticles mRNA vaccine,
DC vaccine, and
ISV: In situ vaccine.

13. A method according to claim 1 where the said Vaccine is combined with a treatment that enhances MHC-II, e.g. a “CIITA treatment” (CIITA is the main transcription factor co-activator of MHC-II) or an “IFN-gamma” treatment.

14. A method according to claim 1 where the said Active Ingredient is combined with other active ingredients in the said Vaccine.

15. A method according to claim 1 where the said Vaccine is combined with a treatment that targets other parts of the T cells, e.g. checkpoint inhibition treatment that targets either of the receptors PD-1 or CTLA-4 on the T cell.

16. A method according to claim 1 where the said Vaccine is administered more than once, and the amount of the said Active Ingredient in the non-first injections is computed adaptively based on measurements of (a) the immune response in terms of e.g. one of or a combination of (1) the expression of the epitopes on MHC-II, (2) the amount of CD4+ cells generated, and, if it is used to treat and not prevent cancer, (3) the amount of cancer cells of a tumor that are killed; and (b) side effects happening as a consequence of the said Vaccine.

17. A method according to claim 1 where the said Vaccine is manufactured and approved with different Active Ingredients, such that there exists a mapping method of allele combinations to a limited set of Vaccines, each with different Active Ingredients, such that the said term “Maximizing The Likelihood” is implemented as a trade-off versus limiting the amount of elements in the said Limited Set of Vaccines, such that all the said vaccines in the Limited Set of Vaccines can be approved while their effects are not severely reduced.


SEQUENCE LISTING (3 sequences)

SEQ ID NO: 1 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 2 - protein, 20 aa, Homo sapiens
SNPPSMVAAGSVVAAVQGLN

SEQ ID NO: 3 - protein, 41 aa, Homo sapiens
LDRFLSLEPVKKSRLQLSNPPSMVAAGSVVAAVQGLNLRSP